(19) United States
(12) Patent Application Publication
(10) Pub. No.: US 2022/0404246 A1
(43) Pub. Date: Dec. 22, 2022

(54) CIRCULATING FLUIDIZED TYPE RAPID SCREENING AND ACCLIMATION DEVICE FOR FUNCTIONAL MICROORGANISMS OF SOIL

(71) Applicant: TSINGHUA UNIVERSITY, BEIJING (CN)

(72) Inventors: Diannan Lu, Beijing (CN); Yakui Chen, Beijing (CN); Zheng Liu, Beijing (CN)

(21) Appl. No.: 17/727,615

(22) Filed: Apr. 22, 2022

(30) Foreign Application Priority Data
Apr. 23, 2021 (CN) 202110440607.1

(51) Int. Cl. G01N 1/40

(52) U.S. Cl. CPC G01N 1/4044; G01N 1/4022; G01N 2001/4088

(57) ABSTRACT

Circulating fluidized type rapid screening and acclimation device for functional microorganisms of soil. The screening and acclimation device includes a fluidized bed body, an air pump and a peristaltic pump. An upper part and a lower part of the fluidized bed body are respectively provided with a liquid outlet device and a liquid inlet device. A porous sieve plate is provided between the liquid outlet device and the fluidized bed body as well as between the liquid inlet device and the fluidized bed body. The liquid outlet device is provided with a liquid outlet and an exhaust port/sampling port. The liquid inlet device is provided with two connectors which are respectively connected to the air pump and the peristaltic pump, and an inlet of the peristaltic pump is connected to the liquid outlet.

### CROSS REFERENCE TO RELATED APPLICATIONS

This patent application claims the benefit and priority of Chinese Patent Application No. 202110440607.1, filed on Apr. 23, 2021, the disclosure of which is incorporated by reference herein in its entirety as part of the present application.

### TECHNICAL FIELD

The present disclosure relates to a circulating fluidized type rapid screening and acclimation device for functional microorganisms of soil, and belongs to the technical field of microorganism screening.

### BACKGROUND ART

Microbial remediation technology has the advantages of being simple in implementation, high in specificity, little in environment damage, and low in cost, which is widely used in the remediation of heavy metals, pesticide residues, organic matter and other pollution problems in the soil. The difficulty and key point of the microbial remediation technology is to find functional microorganisms with high adsorption capacity, high pollutant tolerance and stable adsorption capacity, and how to rapidly screen out effective microorganisms has become the top priority of bio-remediation technology. Traditional screening of microorganisms is mainly done by a selective culture medium dependent method and a liquid phase enrichment method. As there is no suitable selective culture medium for a large number of microbiota, the number of target microbiota obtained by screening and separating is limited, and the traditional separation process is high in cost and long in period. In the liquid phase enrichment method, a triangular flask and a petri dish are generally used to screen the target microorganisms, and in the screening process, the screening difficulty is improved as the triangular flask is fragile, difficult to clean, difficult to operate centrally, large in occupied space, and requires a constant-temperature table concentrator for temperature control; the screening methods are time-consuming and labor-consuming, limited in the number of screened microorganisms, and incapable of meeting the requirements of acquiring and applying the target microorganisms in the current microbial remediation technology, thus the device and method for screening of microorganisms need to be improved.

### SUMMARY

An objective of the present disclosure is to provide a circulating fluidized type rapid screening and acclimation device for functional microorganisms of soil, which solves the problems of high cost, long period, large occupied space, limited number and varieties of screened microorganisms in the traditional microbial screening technology.

The screening and acclimation device provided by the present disclosure can make target soil particles form a fluid-solid contact state, the microorganisms in the soil can be screened and separated under the condition as close to the natural condition as possible without disturbing a soil system and damaging a soil structure, which is conducive to screening a microbial agent capable of adapting to the ecological environment of the soil.

Specifically, the circulating fluidized type rapid screening and acclimation device for functional microorganisms of the soil provided by the present disclosure comprises a fluidized bed body, an air pump, and a peristaltic pump;

An upper part and a lower part of the fluidized bed body are respectively provided with a liquid outlet device and a liquid inlet device, and a porous sieve plate is provided between the liquid outlet device and the fluidized bed body as well as between the liquid inlet device and the fluidized bed body;

The liquid outlet device is provided with a liquid outlet and an exhaust port/sampling port;

The liquid inlet device is provided with two connectors which are respectively connected to the air pump and the peristaltic pump, and an inlet of the peristaltic pump is connected to the liquid outlet.

In the screening and acclimation device, the fluidized bed body is cylindrical and has the advantages of being high in internal space utilization and capable of saving space for placement;

The fluidized bed body can be made of transparent organic glass to facilitate the observation of fluidization condition and guarantee the lighting; and in a case that a light-avoidance culture is required, the fluidized bed can be covered with a cover for shading.

In the screening and acclimation device, the liquid outlet device and the liquid inlet device are both cylindrical.

In the screening and acclimation device, the porous sieve plate is provided with a plurality of round holes, the aperture of the round hole may be 3-5 mm, preferably 4 mm.

In the screening and acclimation device, sealing gaskets are provided between the liquid outlet device and the porous sieve plate, between the porous sieve plate and the fluidized bed body and between the porous sieve plate and the liquid inlet device, and the sealing gaskets play a role in sealing to strengthen the fit between the porous sieve plate and the fluidized bed body.

In the screening and acclimation device, an upper part of the porous sieve plate provided between the fluidized bed body and the liquid inlet device is provided with gauze for preventing fine sand from flowing into the bottom to block a small pipe orifice to affect the circulation and peristalsis.

When the screening and acclimation device of the present disclosure is used, as the fine solid soil particles and liquid in the fluidized bed body form a slurry state, the air is introduced into the fluidized bed body through the air pump to make a gas velocity greater than the free settling velocity of the particles, then the particles are taken out by the gas and after the gas is separated from solids, the solid particles of the soil circularly flow in a fluidized state through the peristaltic pump, thus forming a slurry-state fluidized bed. The slurry flows out from the upper part of the fluidized bed and then flows through the peristaltic pump to be communicated with another connector at the lower end to form circulation. The whole circulating velocity can be regulated through the air pump and the peristaltic pump.

The screening and acclimation device provided by the present disclosure can be used for screening functional microorganisms such as chlorothalonil and quinclorac degradation, straw rotting, heavy metal passivation, organic matter degradation and the like. The screening and acclimation device provided by the present disclosure has obvious advantages over traditional bacteria screening methods, rich microecological structure, strong fungi screening specificity, and simple and convenient sampling.

The screening and acclimation device of the present disclosure has the following beneficial effects:

The screening and acclimation device of the present disclosure is small in occupied space, simple and convenient, and simple to manufacture, the soil circulating fluidization and the soil color change can be observed through the transparent material, and only covering for shading is required if the light-avoidance requirement exists; and the problem that a triangular flask is large in amount, fragile, difficult to clean, large in workload of multiple culture and the like in a traditional liquid-phase enrichment method is solved. When the device is used for bacteria screening, it can be ensured that all soil solid particles are under uniform light, temperature, humidity and fluidization to make the soil be fully decomposed, and the number of microorganisms and the screening rate of the varieties of the microorganisms are increased. In the screening and acclimation process, the water content in the soil is kept constant and is uniformly distributed in the soil, and meanwhile, the water makes the temperature difference of the soil smaller; the soil remains an original structure thereof and is less affected in function; and the presence of oxygen in the circulating liquid allows the oxygen to be distributed in the soil. A soil sample can be taken by only employing circulating liquid, thus making the sampling easy, and meanwhile, the soil itself can be used for sampling. The gas introduced into the device is controllable, and other gases can be used for creating anaerobic or other special environments; the circulating liquid can be replaced at any time to meet different experiment requirements.

### BRIEF DESCRIPTION OF THE DRAWINGS

FIG. 1 is a structure diagram of a circulating fluidized type rapid screening and acclimation device for functional microorganisms of soil of the present disclosure.

FIG. 2A is a front view of a liquid inlet device/a liquid outlet device in the screening and acclimation device of the present disclosure, FIG. 2B is a left sectional view of the liquid inlet device/the liquid outlet device in the screening and acclimation device of the present disclosure, and FIG. 2C is a top view of the liquid inlet device/the liquid outlet device in the screening and acclimation device of the present disclosure.

FIG. 3 is a structure diagram of a porous sieve plate in the screening and acclimation device of the present disclosure, the outside denotes a fixed round hole, and the center of the sieve plate is provided with a plurality of round holes.

FIG. 4 is a structure diagram of a sealing gasket in the screening and acclimation device of the present disclosure.

FIG. 5A is a front view of a fluidized bed body in the screening and acclimation device of the present disclosure, FIG. 5B is a left sectional view of the fluidized bed body in the screening and acclimation device of the present disclosure, and FIG. 5C is a top view of the fluidized bed body in the screening and acclimation device of the present disclosure.

FIG. 6 is a degradation effect of a strain obtained through the screening of a screening and acclimation device of the present disclosure on chlorothalonil.

In the drawings:

1—exhaust port/sampling port; 2—porous sieve plate; 3—sealing gasket; 4—fluidized bed body; 5—air pump; 6—peristaltic pump; 7—liquid outlet device; 8—liquid inlet device.

### DETAILED DESCRIPTION OF THE EMBODIMENTS

The present disclosure is further described below with reference to the accompanying drawings, but the present disclosure is not limited to the following embodiments.

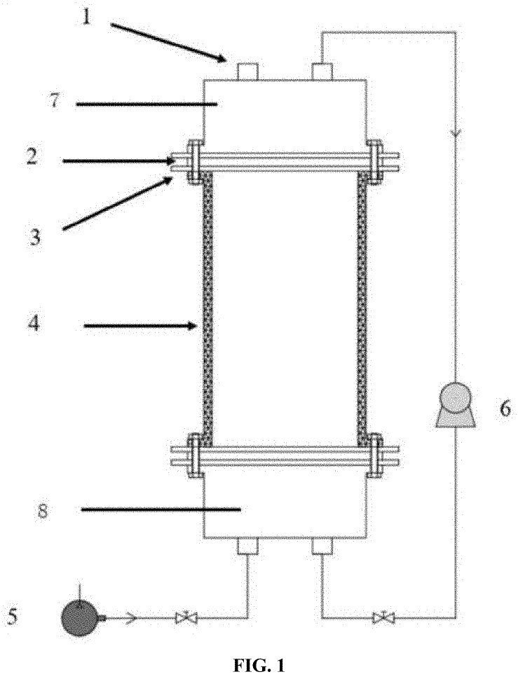

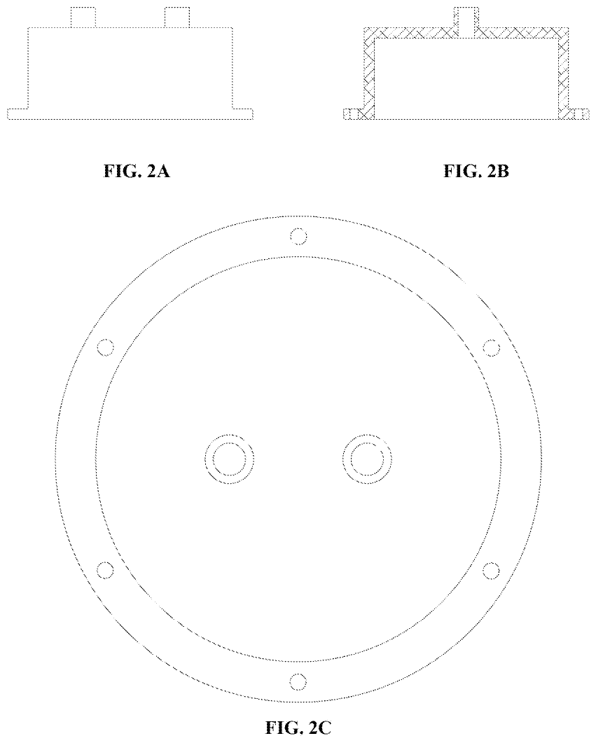

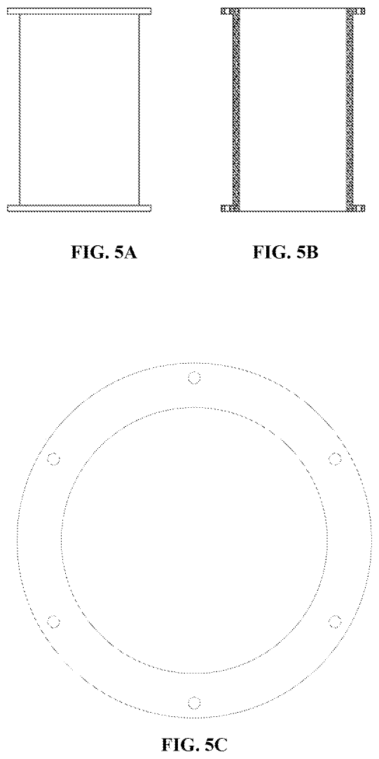

FIG. 1 is a structure diagram of a circulating fluidized type screening and acclimation device, which comprises a fluidized bed body 4, an air pump 5, and a peristaltic pump 6. To facilitate the observation of fluidization, the fluidized bed body 4 is made of transparent organic glass to guarantee the lighting, and can be covered with a cover for shading if light-avoidance culture is required. The liquidized bed is cylindrical, thus the internal space utilization is high, and the placement space can be saved, as shown in FIGS. 5A to 5C. An upper part and a lower part of the fluidized bed body 4 can be respectively provided with a liquid outlet device 7 and a liquid outlet device 8, as shown in FIGS. 2A to 2C, the liquid outlet device 7 is provided with a liquid outlet (not shown in figure) and an exhaust port/sampling port 1, and the liquid outlet is connected to the peristaltic pump 6. The liquid inlet device 8 is provided with two connectors which are respectively connected to the air pump 5 and the peristaltic pump 6. The fine solid soil particles and liquid in the fluidized bed body form a slurry state, the air is introduced into the fluidized bed body through the air pump 5 to make a gas velocity greater than the free settling velocity of the particles, then the particles are taken out by the gas, and after the gas is separated from solids, the solid particles of the soil circularly flow in a fluidized state through the peristaltic pump 6, thus forming a slurry-state fluidized bed.

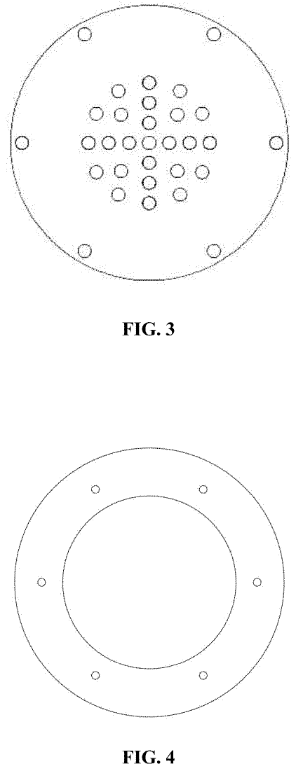

A porous sieve plate 2 is provided between the liquid outlet device 7 and the fluidized bed body 4 as well as between the liquid outlet device 8 and the fluidized bed body 4, and sealing gaskets 3 are provided between the liquid outlet device 7 and the porous sieve plate, between the porous sieve plate 2 and the fluidized bed body 4, and between the porous sieve plate 2 and the liquid inlet device 8 to strengthen the fit between the porous sieve plate 2 and the fluidized bed body 4. The structure diagrams of the porous sieve plate 2 and the sealing gasket 3 are respectively shown in FIG. 3 and FIG. 4. Wherein an upper part of the porous sieve plate 2 provided between the fluidized bed body 4 and the liquid inlet device 8 is provided gauze (not shown in figure) for preventing fine sand from flowing into the bottom to block a small pipe orifice to affect the circulation and peristalsis.

The working process of the circulating fluidized type screening and acclimation device for soil functional microorganisms is as follows:

placing about 200 g of specific soil into the fluidized bed body 4 which is provided with four layers of gauze at the bottom to prevent fine sand from flowing into the bottom to block a small pipe orifice to affect the circulation and peristalsis; and then injecting a prepared liquid nutrient solution to make the soil and the liquid form a slurry state; providing oxygen for the slurry-state fluid through the air pump 5, and enabling the fluid to flow out along with the gas, wherein the pollutant circularly flows in a fluidized state through the peristaltic pump, thus making the water content in the soil be constant and be uniformly distributed in the soil in the screening and acclimation process, and finally forming three-phase equilibrium of gas, liquid, and solid to achieve the acclimation process of the soil.

In the specific acclimation process, the gas displacement of the air pump 5 can be changed, the greater the air pump 5 is, the more sufficient the oxygen supply of the slurry-state fluid is, the activation of the soil microorganisms is facilitated, and the acclimation and bacterial screening process is accelerated. However, over-sized air pump can make the liquid in the liquid outlet device splash, resulting in the increase of the liquid supplementation frequency. Therefore, the optimal gas displacement range is 2-3.2 L/min.

In the specific acclimation process, a rotational speed of the peristaltic pump 6 can be changed, the larger the peristaltic pump is, the faster the circulating velocity of the slurry-state fluid is. However, the circulation effect of the pipeline may be effected due to local lack of oxygen if the peristaltic pump is over-sized, thus the optimal rotational speed range is 170-200 r/min.

The process of screening using the device of the present disclosure is illustrated below by taking the separation of chlorothalonil-degrading bacteria as an example:

1. Culture Medium

inorganic salt liquid culture medium: 1.5 g of NH₄NO₃, 0.5 g of KH₂PO₄, 1.5 g of K₂HPO₄, 1.0 g of NaCl, 0.2 g of MgSO₄.7H₂O, 1000 mL of deionized water, sterilizing at 121° C. for 15 min.

LB solid culture medium: 10 g of peptone, 5 g of yeast, 10 g of NaCl, 25 g of agar, 1000 mL of deionized water, sterilizing at 121° C. for 15 min.

inorganic salt solid culture medium: 1.5 g of NH₄NO₃, 0.5 g of KH₂PO₄, 1.5 g of K₂HPO₄, 1.0 g of NaCl, 0.2 g of MgSO₄.7H₂O, 25 g of agar, 1000 mL of deionized water, sterilizing at 121° C. for 15 min.

PDA culture medium: taking 38 g of potato dextrose agar culture medium and 1000 mL of deionized water, oscillating and mixing, and then sterilizing at 115° C. for 20 min.

Chlorothalonil-containing culture medium: adding a chlorothalonil solution with a certain concentration dissolved in acetone into the liquid or the solid culture medium, and sterilizing at 121° C. for 15 min.

2. Test Soil

harvesting normal soil (15-30 cm) without chlorothalonil applied from the garden of the Science and Technology Park of Xuancheng in Anhui province; and picking out impurities such as straw fragments, broken stones, plastic and roots for later use.

3. Screening of Soil Through Circulating Acclimation Method

sterilizing the fluidized bed body 4, the pipe and the connector at 115° C. for 30 min, then adding the test soil into the fluidized bed body 4, with the soil layer thickness of 5-6 cm; starting the air pump at first, wherein the gas displacement of the air pump is 3.2 L/min, then adding 2 L of sterilized inorganic salt liquid culture medium with the chlorothalonil content of 300 mg/L into the device through the peristaltic pump, wherein the culture medium starts circulation as the peristaltic pump is immersed into the soil layer, and the rotational speed of the peristaltic pump is 300 rpm; and supplementing the circulating liquid at fixed period according to the evaporation condition of the circulating liquid of the inorganic salt culture medium; respectively taking muddy water from the lower connector and the liquid from the upper connector after 2 d, 4 d, 6 d, and 10 d, sequentially diluting by 10, 10², 10³, 10⁴, 10⁵, 10⁶ and 10⁷ times; respectively applying 200 μL of liquid onto the LB solid culture medium, the inorganic salt solid culture medium and the PDA culture medium with the chlorothalonil contents of 100 mg/L, 200 mg/L, 400 mg/L, 500 mg/L, and 600 mg/L, placing the culture mediums in an incubator for inverted culture at 30° C. for 7 d, observing the growth condition of the bacterial colonies, and the picking out strains with different forms to respectively separate and purify, conducting streak separation for multiple times until single colonies are obtained, and refrigerating and storing well-grown pure strains in a refrigerator at 4° C. for later use.

4. Liquid Phase Enrichment Method

taking 10 g of test soil sample to add in a 300 mL conical flask filled with 90 mL of inorganic salt culture medium, and adding an appropriate amount of glass beads; oscillating the conical flask at 30° C. and 170 rpm for 5 h; and centrifuging a soil mixed solution at 3000 rpm for 5 min; inoculating the centrifuged supernatant into the inorganic salt culture medium with the chlorothalonil content of 200 mg/L, and oscillating and culturing at 30° C. and 170 rpm for 3 d; transferring 2 mL of liquid into another conical flask for enrichment culture using a sterile pipette; transferring the liquid for three times to apply the liquid on the LB solid culture medium, the inorganic salt solid culture medium and the PAD culture medium respectively, placing the culture mediums in the incubator for inverted culture at 30° C. for 7 d, observing the growth condition of the bacterial colonies, and then picking out the strains with different forms to respectively separate and purify, conducting streak separation for multiple times until single colonies are obtained, and refrigerating and storing well-grown pure strains in a refrigerator at 4° C. for later use.

5, Screening Result of Chlorothalonil Degradation Strain

screening the chlorothalonil degradation strain from the same source using two methods (the method of the present disclosure and the liquid phase enrichment method), wherein the results show that 17 strains of bacteria and 3 strains of fungi obtained through the screening of a soil circulating acclimation method; and only two strains of bacteria and no fungi are obtained through the screening of the liquid phase enrichment method. Through subsequent strain identification, the result is shown in the following table 1, confirming that the two strains of bacteria obtained through the screening of the liquid-phase enrichment method can also be obtained through the screening of circulating acclimation. Therefore, the strains obtained through the screening of the liquid-phase enrichment method are few in variety and single in bacterial colony; and chlorothalonil degradation strains with rich varieties can be screened by applying a soil circulating acclimation method, the bacterial colonies have rich diversity, and an important foundation is laid for development of efficient chlorothalonil degradation bacteria agent.

TABLE 1
Chlorothalonil degradation bacteria
obtained using different methods

| Screening method | Bacteria | Fungi |
|---|---|---|
| Soil circulating | Enterobacter cloacae, | Cunninghamella |
| acclimation | Burkholderia Symbiont of | bertholletiae, |
| method | Cavelerius saccharivorus, | TUAH-f2 |
| | Burkholderia cepacian, | TUAH-f3 |
| | Bacillus nanhaiensis, Serratia | |
| | marcescens, Pseudomonas | |
| | putida, Burkholderia | |
| | Zhejiangensis, Pseudomonas | |
| | geniculate, Pseudomonas | |
| | monteilii, Pseudomonas | |
| | aeruginosa, TUAH-11, | |
| | Stenotrophomonas maltophilia, | |
| | TUAH-13, Pseudomonas | |
| | dentitrificans, Pseudomonas | |
| | hibiscicola, Burkholderia | |
| | anthina, Stenotrophomonas | |
| | maltophilia | |
| Liquid phase | Burkholderia Symbiont of | — |
| enrichment | Cavelerius | |
| method | Saccharivorus, Burkholderia | |
| | cepacia | |

6. Determination of Degradation Ability of Screened Strains on the Chlorothalonil

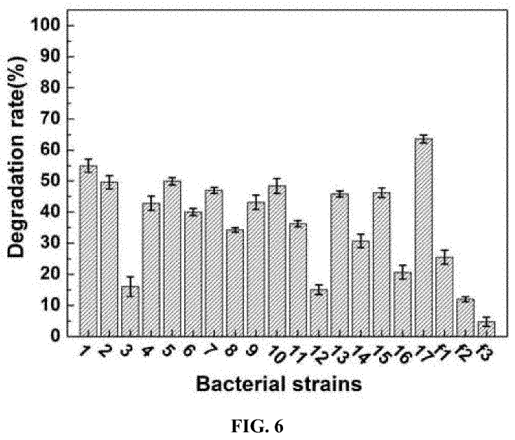

Respectively naming 17 strains of bacteria obtained through screening as TUAH-1 to TUAH-17, and naming three strains of fungi as TUAH-f1, TUAH-f2, and TUAH-f3, wherein the degradation rate of the strain to 20 mg/L chlorothalonil for two days is as shown in FIG. 6.

The result shows that the strains obtained through the screening of the soil circulating acclimation method using the device of the present disclosure have degradation effect on Chlorothalonil, and the degradation rate is up to 63.6%, which has laid a good foundation for the subsequent practical degradation of Chlorothalonil in the field.

The screening and acclimation device of the present disclosure is simple in sampling in later period, the gas introduction in the device is controllable, the acclimation flow rate is controllable, and other gases can be used for creating anaerobic or other special environments; and the circulating liquid can be replaced at any time to meet different experiment requirements.

In the circulating acclimation process, the advantages of simple operation, energy conservation, emission reduction, low energy consumption and low cost of the device are fully reflected.

1. A circulating fluidized type rapid screening and acclimation device for functional microorganisms of soil, comprising:
a fluidized bed body, an air pump, and a peristaltic pump;
an upper part and a lower part of the fluidized bed body are respectively provided with a liquid outlet device and a liquid inlet device, and a porous sieve plate is provided between the liquid outlet device and the fluidized bed body as well as between the liquid inlet device and the fluidized bed body;
the liquid outlet device is provided with a liquid outlet and an exhaust port/sampling port;
the liquid inlet device is provided with two connectors which are respectively connected to the air pump and the peristaltic pump, and an inlet of the peristaltic pump is connected to the liquid outlet.

2. The screening and acclimation device according to claim 1, wherein the fluidized bed is cylindrical.

3. The screening and acclimation device according to claim 1, wherein the fluidized bed is made of transparent organic glass.

4. The screening and acclimation device according to claim 2, wherein the fluidized bed is made of transparent organic glass.

5. The screening and acclimation device according to claim 1, wherein the liquid outlet device and the liquid inlet device are both cylindrical.

6. The screening and acclimation device according to claim 2, wherein the liquid outlet device and the liquid inlet device are both cylindrical.

7. The screening and acclimation device according to claim 3, wherein the liquid outlet device and the liquid inlet device are both cylindrical.

8. The screening and acclimation device according to claim 4, wherein the liquid outlet device and the liquid inlet device are both cylindrical.

9. The screening and acclimation device according to claim 1 wherein the porous sieve plate is provided with a plurality of round holes, and the round hole has an aperture of 3-5 mm.

10. The screening and acclimation device according to claim 2 wherein the porous sieve plate is provided with a plurality of round holes, and the round hole has an aperture of 3-5 mm.

11. The screening and acclimation device according to claim 3, wherein the porous sieve plate is provided with a plurality of round holes, and the round hole has an aperture of 3-5 mm.

12. The screening and acclimation device according to claim 4, wherein the porous sieve plate is provided with a plurality of round holes, and the round hole has an aperture of 3-5 mm.

13. The screening and acclimation device according to claim 5, wherein the porous sieve plate is provided with a plurality of round holes, and the round hole has an aperture of 3-5 mm.

14. The screening and acclimation device according to claim 6, wherein the porous sieve plate is provided with a plurality of round holes, and the round hole has an aperture of 3-5 mm.

15. The screening and acclimation device according to claim 7, wherein the porous sieve plate is provided with a plurality of round holes, and the round hole has an aperture of 3-5 mm.

16. The screening and acclimation device according to claim 8, wherein the porous sieve plate is provided with a plurality of round holes, and the round hole has an aperture of 3-5 mm.

17. The screening and acclimation device according to claim 1, wherein the sealing gaskets are provided between the liquid outlet device and the porous sieve plate, between the porous sieve plate and the fluidized bed body, and between the porous sieve plate and the liquid inlet device.

18. The screening and acclimation device according to claim 2, wherein sealing gaskets are provided between the liquid outlet device and the porous sieve plate, between the porous sieve plate and the fluidized bed body, and between the porous sieve plate and the liquid inlet device.

19. The screening and acclimation device according to claim 3, wherein sealing gaskets are provided between the liquid outlet device and the porous sieve plate, between the porous sieve plate and the fluidized bed body, and between the porous sieve plate and the liquid inlet device.

20. The screening and acclimation device according to claim 1, wherein an upper part of the porous sieve provided between the fluidized bed and the liquid inlet device is provided with gause.
